(19) United States
(12) Patent Application Publication
(10) Pub. No.: US 2015/0352006 A1
(43) Pub. Date: Dec. 10, 2015

(54) Indirect Bird-Pecking Moxibustion Device that is Portable Automatically Height Adjustable and Horizontally Movable

(71) Applicant: Yuping Sun, Jinan Shandong (CN)

(72) Inventor: Yuping Sun, Jinan Shandong (CN)

(21) Appl. No.: 14/566,292

(22) Filed: Dec. 10, 2014

(30) Foreign Application Priority Data
Jun. 9, 2014 (CN) 201410249187.9

(51) Int. Cl. A61H 39/06; A61F 7/02

(52) U.S. Cl. CPC A61H 39/06; A61F 7/02; A61H 2201/0157; A61F 2007/0207; A61H 2201/1664

(57) ABSTRACT

An indirect bird-pecking moxibustion device that is portable, automatically height adjustable, and horizontally movable. The device comprises a hollow tube adapted to hold an ignited moxa cone over an acupuncture point, an adjustment system that enables the moxa cone to be placed at different horizontal locations above the different targeted acupuncture points, and a mechanism connected to the hollow tube reciprocally and vertically moving the hollow tube in relation to the acupuncture point.

The present application hereby claims priority to the Chinese application No. 201410249187.9, filed on Jun. 9, 2014.

### TECHNICAL FIELD

The present invention belongs to the medical device technical field. More specifically, the present invention is an indirect bird-pecking moxibustion device that is portable, automatically height adjustable and horizontally movable.

### BACKGROUND

Moxibustion is a traditional Chinese medicine therapy using moxa made from dried mugwort (Artemisia argyi). Although available scientific evidence does not support claims that moxibustion is effective in preventing or treating cancer or any other disease, it plays an important role in the traditional medical systems of China, Japan, Korea, Vietnam, and Mongolia. Suppliers usually age the mugwort and grind it up to a fluff. Practitioners burn the fluff or process it further into a cigar-shaped stick, known as a moxa cone.

There are several methods of moxibustion. Three of them are direct scarring, direct non-scarring, and indirect moxibustion. Direct scarring moxibustion places a small cone of moxa on the skin at an acupuncture point and burns it until the skin blisters, which then scars after it heals. Direct non-scarring moxibustion removes the burning moxa before the skin burns enough to scar, unless the burning moxa is left on the skin too long. Indirect moxibustion requires a practitioner to hold a moxa cone near an acupuncture point to heat the skin, or hold it on an acupuncture needle inserted in the skin to heat the needle. Bird-pecking moxibustion refers to a type of indirect moxibustion that requires a practitioner to hold a burning moxa cone with the burning end over an acupuncture point, and move the cone up and down in a way similar to bird pecking to give the patient a moxibustion with a stronger but intermittent hot stimulation. There is no fixed distance between the moxa cone and the patient to be treated by moxibustion.

The world Health Organization (WHO) published a A Proposed Standard International Acupuncture Nomenclature Report in 1991, listing 361 classical acupuncture points organized according to the fourteen meridians, eight extra meridians, 48 extra points, and scalp acupuncture points, and published Standard Acupuncture Nomenclature in 1993, focused on the 361 classical acupuncture points. According to the disclosed locations, many of the acupuncture points are close to each other and within a line. The most well known examples are the acupuncture points located along the governing vessel and the conception vessel. Take the conception vessel for example. It starts from the inside of the lower abdomen and comes out from the perineum. It goes anteriorly to the pubic region and ascends along the interior side of the abdomen, passes through Guanyan (CV4) and reaches the throat. Ascending further, it curves around the lips, passes through the cheek and enters the infraorbital region Chengqi (ST1). The acupuncture points along the conception vessel are CV1Huiyin, CV2Qugu, CV3Zhongji, CV4Guanyuan, CV5Shimen, CV6Qihai, CV7Yinjiao, CV8Shenque, CV9Shuifen, CV1OXiawan, CV11Jianli, CV12Zhongwan, CV13Shangwan, CV14Juque, CV15Jiuwei, CV16Zhongting, CV17Danzhong, CV18Yutang, CV19Zigong, CV20Huagai, CV21Xuanji, CV22Tiantu, CV23Lianquan, CV24Chengjiang. It is believed that when treating patients who have stomach problems, practitioners can perform moxibustion of CV16Zhongting, CV13Shangwan, and CV12Zhongwan. When treating patients with enuresis, practitioners can perform moxibustion on CV6Qihai and CV4Guanyuan.

When treating patients who suffer from gynecological, digestive system, urinary system, cardiovascular, high blood pressure, endocrine, or orthopedic diseases, or when helping people in enhancing immunity, traditionally it requires a practitioner to hold a lighted moxa cone in his or her hand pointed to related acupuncture points to complete the indirect moxibustion. The practitioner also needs to adjust the distance between the moxa cone and the patient to achieve the most comfortable temperature. These procedures make moxibustion difficult and labor intensive. The treatment has to be one-on-one, which is inefficient. During the process, the patient's activities are limited. Most of the time, he or she has to remain still for a long period of time. Moreover, when multiple patients are treated in a single room, the accumulated smoke will be harmful to the patients. All of those shortcomings mentioned above make the treatment unpleasant.

### SUMMARY

In order to overcome those aforementioned shortcomings, the present invention provides an indirect bird-pecking moxibustion device that is portable, automatically height adjustable, and horizontally movable. It comprises a hollow tube adapted to hold an ignited moxa cone over an acupuncture point, an adjustment system that enables the moxa cone to be placed at different horizontal locations above the different targeted acupuncture points, and a mechanism connected to the hollow tube reciprocally and vertically moving the hollow tube in relation to the acupuncture point.

In accordance with the invention, patients can complete moxibustion treatments while lying down or standing up. When there is no need to eject smoke generated while burning moxa cone, patients can even be treated while walking The treatment can be completed either at home, or in a hospital. It will reduce the labor intensity, and also enable practitioners to apply multiple treatments on different patients. The process can also be controlled by computer programs as well.

### DESCRIPTION OF DRAWINGS

FIG. 1 is a perspective schematic view of the invention.

FIG. 2 is an enlarged sectional view of FIG. 1 taken along line A-A in FIG. 1.

FIG. 3 is an upward view taken from B in FIG. 1.

FIG. 4 is an enlarged sectional view of FIG. 1 taken along line C-C in FIG. 1

### DETAILED DESCRIPTION

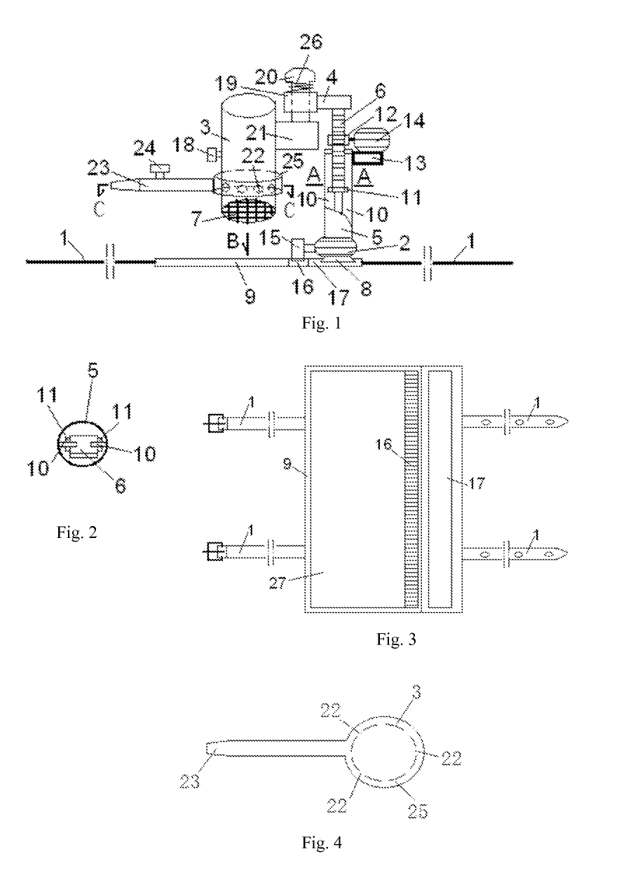

Referring to FIGS. 1, 2 and 3, a moxa cone holding tube 3 connects to the left side of a fixed block 21. The top of the fixed block 21 connects to the bottom of a moving core 20. The moving core 20 inserts into a hollow portion of an electromagnetic bobbin 19. A return spring 26 urges the moving core 20 upward. The electromagnetic bobbin 19 connects to the left side of a horizontal support frame 4. The underside of the horizontal support frame 4 connects to the top of a vertically movable lifting and lowering rack 6. The vertically movable lifting and lowering rack 6 is inserted between two slides 10 located in a tube 5. A limit block 11 is affixed to the bottom of the vertically movable lifting and lowering rack 6. A supporting frame 13 is affixed to the tube 5. A low speed motor 14 that contains a first gear 12 is affixed to the supporting frame 13. The first gear 12 meshes with the vertically movable lifting and lowering rack 6.

The bottom of the tube 5 is affixed to the top of a stepping motor 2. The bottom of the stepping motor 2 connects to a dovetail block 8. The dovetail block 8 inserts into a dovetail slot 17. The dovetail slot 17 and an acupuncture channel 27 (FIG. 3) are located on the opposite side of an adjustment frame 9. A horizontally disposed rack 16 is also located on the adjustment frame 9 and in between the dovetail slot 17 and the acupuncture channel 27. Positioning belts 1 are attached to both sides of the adjustment frame 9. A second gear 15 is affixed to the shaft of the stepping motor 2. The second gear 15 meshes with a horizontally disposed rack 16.

Smoke holes 22 are located on the surface of the moxa cone holding tube 3. An evacuation annular tube 25 (FIG. 4) surrounds the moxa cone holding tube 3 at its lower half, and connects to the moxa cone holding tube 3 through smoke holes 22. The evacuation plug tube 23 is equipped with a switch 24 and also connects to the evacuation annular tube 25. One or more fastening screws 18 are attached to the moxa cone holding tube 3. An ash receiver 7 is located at the bottom of the moxa cone holding tube 3.

When treating patients with gynecological, digestive system, urinary system, cardiovascular, high blood pressure, endocrine, or orthopedic diseases, or when helping people in enhancing immunity, a practitioner will insert an ignited moxa cone into the moxa cone holding tube 3, with the lighted end of the moxa cone close to the ash receiver 7. After locating the targeted acupuncture points, the practitioner will place the acupuncture channel 27 located on the adjustment frame 9 around the targeted acupuncture points area, and use the positioning belts 1 to fasten and fix the device to a patient's body. The practitioner will control the low speed motor 14 to rotate the first gear 12. As a result, the first gear 12 will move the vertically movable lifting and lowering rack 6 to a position that locates the moxa cone at a height that a patient feels comfortable with. If a patient is in need of a stronger treatment, the practitioner can remove the ash receiver 7, and use the fastening screws 18 to directly locate the moxa cone to the targeted acupuncture point area. The limit block 11 will prevent the vertically movable lifting and lowering rack 6 from sliding out of the tube 5.

When the electromagnetic bobbin 19 is provided with pulsed electricity, the moving core 20 and the return spring 26 will reciprocate up and down to allow the moxa cone to complete a bird-pecking moxibustion on the skin of the patient. When multiple targeted acupuncture points are located close to each other and within a line, a practitioner will control the stepping motor 2 to rotate the second gear 15, so that under the assistance of the dovetail block 8 and dovetail slot 17, the entire device will be moved horizontally along the horizontally disposed rack 16. The practitioner can repeat this operation to complete a series of moxibustions along a line.

The evacuation plug tube 23 is connected to the evacuation annular tube 25, so that when burning the moxa cone to treat patients, smoke will be ejected out of the room through the smoke holes 22, evacuation annular tube 25 and evacuation plug tube 23. By controlling the switch 24, the practitioner will be able to open or close the airway inside of the plug tube 19 to manage smoke ejection. Because the smoke holes 22 are designed to be located close to the bottom of the moxa cone holding tube 3, they will catch all the generated smoke, and ensure oxygen flow to let the moxa cone fully combust and reduce smoke.

What is claimed is:

1. A moxibustion device for delivering moxa at an acupuncture point, comprising:
a hollow tube adapted to hold an ignited moxa cone over an acupuncture point, an adjustment system enabling the moxa cone to be selectively located at different acupuncture point locations and above different targeted acupuncture points, and a mechanism connected to the hollow tube reciprocally moving the hollow tube in relation to the acupuncture point.

2. A moxibustion device according to claim 1, wherein the mechanism connected to the hollow tube that reciprocally moves the hollow tube comprises a linkage and a low speed motor that provides varying vertical placements of the hollow tube.

3. A moxibustion device according to claim 2, wherein the linkage comprises a vertically movable lifting and lowering rack inserted between two slides located in a tube for positioning the moxa cone over a selected acupuncture point.

4. A moxibustion device according to claim 3, wherein the linkage comprises a limit block fixed to the bottom of the vertically movable lifting and lowering rack to prevent the rack from sliding out of the tube.

5. A moxibustion device according to claim 3, wherein the linkage comprises a horizontal support frame that connects the top of the vertically movable lifting and lowering rack to an electromagnetic bobbin.

6. A moxibustion device according to claim 5, wherein the linkage comprises a moving core that inserts into the electromagnetic bobbin.

7. A moxibustion device according to claim 6, wherein the linkage comprises a return spring between the moving core and the electromagnetic bobbin, the return spring returning the moving core to an upper position during operation of the bobbin.

8. A moxibustion device according to claim 6, wherein the linkage comprises a fixed block connecting the bottom of the moving core and the hollow tube.

9. A moxibustion device according to claim 1, wherein the adjustment system comprises a stepping motor that connects to the bottom of the tube and is operatively connected to an adjustment frame.

10. A moxibustion device according to claim 9, wherein a gear is affixed to a shaft of the stepping motor and said gear meshes with a rack located on the adjustment frame.

11. A moxibustion device according to claim 9, wherein the adjustment frame comprises an acupuncture channel and a dovetail slot, each located on opposite sides of the adjustment frame.

12. A moxibustion device according to claim 11, wherein the bottom of the stepping motor connects to a dovetail block.

13. A moxibustion device according to claim 12, wherein the dovetail block inserts into the dovetail slot to assist the hollow tube to move horizontally along the adjustment frame.

14. A moxibustion device according to claim 1, further comprising an exhaust system to eject smoke from the hollow tube.

15. A moxibustion device according to claim 14, wherein the exhaust system connected to the hollow tube comprises smoke holes located on the surface of the hollow tube, an evacuation plug tube equipped with a switch, and an evacuation annular tube that connects to the hollow tube through the smoke holes and the evacuation plug tube.

16. A moxibustion device according to claim 1, further comprising one or more fastening screws set in the hollow tube.

17. A moxibustion device according to claim 1, further comprising an ash receiver set at the bottom of the hollow tube.

18. A moxibustion device according to claim 1, further comprising an external fixation device.

19. A moxibustion device according to claim 18, wherein the external fixation device comprises positioning belts connected to the adjustment frame.
